(19) United States
(12) Patent Application Publication
(10) Pub. No.: US 2004/0166159 A1
(43) Pub. Date: Aug. 26, 2004

(54) Pharmaceutical dosage forms having immediate and controlled release properties that contain an aromatic amino acid decarboxylase inhibitor and levodopa

(76) Inventors: Chien-Hsuan Han, Sunnyvale, CA (US); Ann Hsu, Los Altos Hills, CA (US); Larry Hsu, Los Altos Hills, CA (US)

Correspondence Address: PRESTON GATES ELLIS & ROUVELAS MEEDS LLP, 1735 NEW YORK AVENUE, NW, SUITE 500, WASHINGTON, DC, 20006, US

(21) Appl. No.: 10/787,417

(22) Filed: Feb. 27, 2004

Related U.S. Application Data
(63) Continuation-in-part of application No. 10/158,412, filed on May 29, 2002.

(51) Int. Cl. A61K 31/198; A61K 9/22

(52) U.S. Cl. 424/468; 514/567

(57) ABSTRACT

The present invention relates generally to pharmaceutical dosage forms having immediate and controlled release properties that contain an aromatic amino acid decarboxylase (AAAD) inhibitor (such as carbidopa), levodopa, and optionally a cathecol-O-methyltransferase (COMT) inhibitor, for the treatment of medical conditions associated with reduced dopamine levels in a patient's brain.

### CROSS-REFERENCE TO RELATED APPLICATION

This application is a continuation-in-part, and claims the benefit under 35 U.S.C. § 120, of U.S. patent application Ser. No. 10/158,412, filed on May 29, 2002, by Chien-Hsuan Han et al., entitled Combination Immediate Release Sustained Release Levodopa/Carbidopa Dosage Forms, which is incorporated herein by reference in its entirety.

### BACKGROUND OF THE INVENTION

The present invention relates generally to pharmaceutical dosage forms having immediate and controlled release properties that contain an aromatic amino acid decarboxylase (AAAD) inhibitor (such as carbidopa), levodopa, and optionally a cathecol-O-methyltransferase (COMT) inhibitor, for the treatment of medical conditions associated with reduced dopamine levels in a patient's brain.

Parkinson's disease is a progressive, neurodegenerative disorder of the extrapyramidal nervous system that affects the mobility and control of the skeletal muscular system. Its medical signs include resting tremor, rigidity, and bradykinetic movements. A symptom of Parkinson's disease is a reduced dopamine level in the patient's brain.

The administration of dopamine is not effective in treating Parkinson's disease because dopamine does not cross the blood brain barrier. Rather, patients with Parkinson's disease are typically administered levodopa, the metabolic precursor of dopamine. Levodopa crosses the blood brain barrier and is rapidly converted to dopamine, thereby alleviating the symptoms of Parkinson's disease caused by reduced levels of dopamine. However, treatment with levodopa is problematic because of its rapid decarboxylation by tissues other than the brain. Thus, when levodopa is administered alone, large doses are required because only a small, effective amount of levodopa is available upon transport to the brain.

Patients undergoing levodopa therapy for Parkinson's disease frequently can develop motor fluctuations characterized by end-of-dose failure, peak dose dyskinesia and akinesia. An advanced form of motor fluctuations is known as the “on-off effect” in which the patient suffers from unpredictable swings from mobility to immobility. This on-off effect might be minimized in some patients with a treatment regimen that produces narrow ranges of plasma levels of levodopa.

Carbidopa inhibits the decarboxylation of levodopa by a patient's body tissues outside of the brain. Small doses of carbidopa administered in conjunction with levodopa allow larger, effective amounts of levodopa to reach the brain and be converted to dopamine. For example, carbidopa reduces the amount of levodopa required to produce a given response by about 75% and, when administered in conjunction with levodopa, increases plasma levels and the plasma half life of levodopa. A combination therapy of carbidopa and levodopa facilitates the administration of smaller doses of levodopa, which can provide a concomitant reduction of any side effects.

Pharmaceutical combinations of carbidopa and levodopa are available for the treatment of Parkinson's disease. Such products include SINEMET® tablets and SINEMET® CR sustained release tablets (Bristol-Myers Squibb Co.). In addition, pharmaceutical products are also available that incorporate other AAAD inhibitors in combination with levodopa, such as the Madopark® products (Roche), which include benserazide. The controlled release formulations allow for the continuous release of drug over a prolonged period in an attempt to maintain tight levodopa plasma ranges. However, the use of controlled release dosage forms are problematic in that many patients with Parkinson's disease wake up in the morning having little or no mobility because the previous dose taken the day or evening before has worn off. Once the previous dose has worn off, such patients are usually unwilling or unable to wait for the extended period of time required for a controlled release dosage form to deliver the appropriate plasma levels of levodopa. Similarly, the use of immediate release formulations alone is problematic in that it requires more frequent dosing and are associated with more fluctuating plasma levodopa concentrations.

Another adjunct to the treatment of Parkinson's disease through pharmaceutical combinations of carbidopa and levodopa has been the use of a COMT inhibitor, such as entacapone. When entacapone is given in conjunction with levodopa and an AAAD inhibitor, such as carbidopa, plasma levels of levodopa are greater and more sustained than after administration of levodopa and an AAAD inhibitor alone. For example, when 200 mg of entacapone is administered together with a combination of carbidopa and levodopa, the area under the curve (AUC) of levodopa can be increased by about 35%, and the elimination half life of levodopa is prolonged from 1.3 hours to 2.4 hours. Pharmaceutical combinations of carbidopa, levodopa, and entacapone are available for the treatment of Parkinson's disease. Such products include STALEVO® (Novartis Pharmaceuticals Corp.).

However, a continuing need remains for pharmaceutical dosage forms having immediate and controlled release properties that contain an AAAD inhibitor, (such as carbidopa), levodopa, and optionally a COMT inhibitor, for the treatment of medical conditions associated with reduced dopamine levels in a patient's brain that facilitate the improved administration of levodopa to patients with Parkinson's disease by narrowing blood plasma ranges of levodopa and reducing side effects, among other things. These and other objectives are accomplished by the present invention.

### BRIEF SUMMARY OF THE INVENTION

The present invention relates generally to pharmaceutical dosage forms comprising an immediate release component and a controlled release component that contain an AAAD inhibitor (such as carbidopa), levodopa, and optionally a cathecol-O-methyltransferase (COMT) inhibitor. These dosage forms can be used in the treatment of medical conditions associated with reduced dopamine levels in a patient's brain.

For example, the pharmaceutical dosage forms of the present invention may include an immediate release component and said controlled release component wherein each comprises an AAAD inhibitor and levodopa in a ratio of from about 1:1 to about 1:50; wherein the immediate release component exhibits an in vitro dissolution profile comprising at least about 10% levodopa release after 15 minutes and at least about 60% levodopa release after I hour; and wherein the controlled release component exhibits an in vitro dissolution profile comprising from about 10% to about 60% levodopa release after 1 hour minutes, from about 20% to about 80% levodopa release after 2 hours, and at least about 30% levodopa release after 6 hours. The pharmaceutical dosage forms of the present invention may also exhibit an in vivo plasma profile comprising a levodopa release peak from about 6 minutes to about 6 hours after administration to a fasting patient. Furthermore, the pharmaceutical dosage forms of the present invention may include a COMT inhibitor in the immediate release component, the controlled release component, or both.

### BRIEF DESCRIPTION OF THE SEVERAL VIEWS OF THE DRAWINGS

FIG. 1 is a graph of the dissolution profiles of carbidopa/levodopa immediate release (IR) 25/100 mg formulations PX03002 and PX03102 according to measurements under the USP paddle method of 50 rpm in 900 ml acetate buffer at pH 4 at 37° C.

FIG. 2 is a graph of the dissolution profile of a carbidopa/levodopa controlled release (CR) 50/200 mg formulation PX00502 according to measurements under the USP paddle method of 50 rpm in 900 ml acetate buffer at pH 4 at 37° C.

FIG. 3 is a graph of the dissolution profiles of carbidopa/levodopa 75/300 mg formulations PX03602 and PX04002 according to measurements under the USP paddle method of 50 rpm in 900 ml acetate buffer at pH 4 at 37° C.

FIG. 4 is a graph of the dissolution profiles of carbidopa/levodopa immediate release (IR) 25/100 mg formulations PX00102, PX02001, and Brand K5370 according to measurements under the USP paddle method of 50 rpm in 900 ml at pH 1.2(0.1 N HCl) at 37° C.

FIG. 5 is a graph of the dissolution profiles of carbidopa/levodopa controlled release (CR) 50/200 mg formulations PX00302, PX00502, and Brand 01023 according to measurements under the USP paddle method of 50 rpm in 900 ml at pH 1.2 (0.1 N HCl) at 37° C.

FIG. 6 is a graph of the dissolution profiles of carbidopa/levodopa formulations PX03602 (controlled release, 75/300 mg), PX04002 (controlled release, 75/300 mg), Brand K5370 (immediate release, 25/100 mg), and Brand 01023 (controlled release, 50/200 mg) according to measurements under the USP paddle method of 50 rpm in 900 ml at pH 1.2 (0.1 N HCl) at 37° C.

### DETAILED DESCRIPTION OF THE INVENTION

The present invention relates to pharmaceutical dosage forms comprising an immediate release component and a controlled release component that contain an AAAD inhibitor (such as carbidopa), levodopa, and optionally a cathecol-O-methyltransferase (COMT) inhibitor. These dosage forms can be used in the treatment of medical conditions associated with reduced dopamine levels in a patient's brain. Such conditions include symptoms, pathologies, or diseases such as neurological or movement disorders associated with restless leg syndrome, Parkinson's disease and secondary parkinsonism, Huntingdon's disease, Shy-Drager syndrome, as well as those resulting from brain injury attributable to carbon monoxide or manganese intoxication.

An embodiment of the present invention may be a pharmaceutical dosage form comprising an immediate release and a controlled release component, wherein the immediate release component comprises an AAAD inhibitor (such as carbidopa) and levodopa (and/or 3-hydroxy-L-tyrosine ethyl ester) in a ratio of from about 1:1 to about 1:50 (preferably from about 1:1 to about 1:10, more preferably from about 1:1 to about 1:5, and even more preferably from about 1:1 to about 1:4) plus an optional COMT inhibitor, and/or wherein the controlled release component comprises an AAAD inhibitor (such as carbidopa) and levodopa (and/or 3-hydroxy-L-tyrosine ethyl ester) in a ratio of from about 1:1 to about 1:50 (preferably from about 1:1 to about 1:10, more preferably from about 1:1 to about 1:5, and even more preferably from about 1:1 to about 1:4) plus an optional COMT inhibitor; wherein the immediate release component exhibits an in vitro dissolution profile comprising at least about 10% levodopa release after 15 minutes (or after 30 minutes) and at least about 60% (preferably 95%) levodopa release after 1 hour; and wherein the controlled release component exhibits an in vitro dissolution profile comprising from about 10% to about 60% levodopa release after I hour, from about 20% to about 80% levodopa release after 2 hours, and at least about 30% levodopa release after 6 hours.

Another embodiment of the present invention may be a pharmaceutical dosage form that exhibits an in vivo plasma profile comprising a levodopa release peak from about 6 minutes to about 6 hours (preferably from about 6 minutes to about 5 hours) after administration to a fasting patient.

One other embodiment of the present invention may be a pharmaceutical dosage form that exhibits an in vivo plasma profile comprising a COMT inhibitor release peak from about 6 minutes to about 6 hours (preferably from about 6 minutes to about 5 hours) after administration to a fasting patient. In this regard, the pharmaceutical dosage form may comprise up to about 1000 mg (preferably from about 20 mg to about 500 mg, more preferably from about 50 mg to about 500 mg, and even more preferably from about 100 mg to about 200 mg) COMT inhibitor. Further in this regard, the COMT inhibitor may be contained within the immediate release component only, within the controlled release component only, or within both the immediate release and the controlled release components. Also, the COMT inhibitor may be CGP-28014, entacapone, and tolcapone.

Yet another embodiment of the present invention may be a pharmaceutical dosage form that further comprises one or more drugs selected from the group consisting of anti-cholinergics, beta 2-agonists, cyclooxygenase-2 (COX-2) inhibitors, dopamine receptor agonists, monoamine oxidase (MAO) inhibitors, opiate delta receptor agonists, opiate delta receptor antagonists, and N-methyl-D-aspartate (NMDA) antagonists. One other embodiment may also include one or more drugs selected from the group consisting of albuterol, alpha-lipoic acid, amantadine, andropinirole, apomorphine, baclofen, biperiden, benztropine, bromocriptine, budipine, cabergoline, clozapine, deprenyl, dextromethorphan, dihydroergokryptine, dihydrolipoic acid, eliprodil, eptastigmine, ergoline, formoterol, galanthamine, lazabemide, lysuride, mazindol, memantine, mofegiline, orphenadrine, pergolide, pirbuterol, pramipexole, propentofylline, procyclidine, rasagiline, remacemide, riluzole, rimantadine, ropinirole, salmeterol, selegiline, spheramine, terguride, and trihexyphenidyl.

In another embodiment of the present invention, the pharmaceutical dosage form may include an immediate release component that comprises from about 2.5 mg to about 75 mg of carbidopa and from about 25 mg to about 300 mg levodopa. In yet another embodiment of the present invention, the pharmaceutical dosage form may include a controlled release component that comprises from about 2.5 mg to about 200 mg of (preferably from about 5 mg to about 150 mg, and more preferably from about 12.5 mg to about 50 mg) carbidopa and from 25 mg to about 600 mg (preferably from about 50 mg to about 500 mg, and more preferably from about 50 mg to about 200 mg) levodopa.

In other embodiments of the present invention, the pharmaceutical dosage form may be a particle, a tablet, or a layered tablet.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms “a,” “an,” and “the” include the plural reference unless the context clearly indicates otherwise. Thus, for example, the reference to a profile is a reference to one or more such profiles, including equivalents thereof known to those skilled in the art. Other than in the operating examples, or where otherwise indicated, all numbers expressing quantities of ingredients or reaction conditions used herein should be understood as modified in all instances by the term “about.” The term “about” when used in connection with percentages can mean +1%.

All patents and other publications identified are incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason.

Unless defined otherwise, all technical and scientific terms used herein have the same meaning as those commonly understood to one of ordinary skill in the art to which this invention pertains. Although any known methods, devices, and materials may be used in the practice or testing of the invention, the preferred methods, devices, and materials in this regard are described here.

AAAD inhibitors known in the art include carbidopa, benserazide, alpha-monofluoromethyldopa, and 3-hydroxybenzylhydrazine. Carbidopa is a known AAAD inhibitor having the formula (−)-L-(α-hydrazino-(α-methyl-β-(3,4-dihydroxybenzene) propanoic acid monohydrate. Levodopa is a know aromatic amino acid precursor of dopamine having the formula (−)-L-α-amino-β-(3,4-dihydroxybenzene) propanoic acid. COMT inhibitors include CGP-28014, entacapone, and tolcapone.

U.S. Pat. No. 6,238,699 (Rubin) describes certain combination immediate release and controlled release carbidopa and levodopa dosage forms. The use of entacapone has been studied in conjunction with carbidopa/levodopa therapy in patients with Parkinson's disease. See Ahtila et al., “Effect of Entacapone, A COMT Inhibitor, on the Pharmacokinetics and Metabolism of Levodopa After Administration of Controlled release Levodopa-Carbidopa in Volunteers,” Clinical Neuropharmacology, 18(1), 46-57 (1995). U.S. Pat. No. 6,500,867 (Virkki et al.) discloses formulations containing levodopa, carbidopa, and entacapone.

For purposes of the present invention the term “controlled release” refers to part of all of a dosage form that can release one or more active pharmaceutical agents over a prolonged period of time (i.e., over a period of more than 1 hour). The characteristic of controlled release (CR) may also be referred to as sustained release (SR), prolonged release (PR), or extended release (ER). When used in association with the dissolution profiles discussed herein, the term “controlled release” refers to that portion of a dosage form according to the present invention that delivers active agent over a period of time greater than 1 hour.

“Immediate release” refers to part or all of a dosage form that releases active agent substantially immediately upon contact with gastric juices and that results in substantially complete dissolution within about 1 hour. The characteristic of immediate release (IR) may also be referred to as instant release (IR). When used in association with the dissolution profiles discussed herein, the term “immediate release” refers to that portion of a dosage form according to the present invention that delivers active agent over a period of time less than 1 hour.

Initial peak plasma level refers to the first rise in blood plasma level of active agent and may be followed by one or more additional peaks, one of which may be referred to as C_{MAX}.

The USP paddle method refers to the Paddle and Basket Method as described in United States Pharmacopoeia, Edition XXII (1990). In particular, the USP paddle method of 50 rpm in 900 ml aqueous buffer at pH 4 at 37° C. may be used to determine the in vitro dissolution profiles according to the present invention.

As used herein, the term “patient” means any mammal including humans.

The drugs suitable for use in the pharmaceutical dosage forms according to the present invention include the specified chemical compound as well as salts, analogues, metabolites, derivatives, solvates, and prodrugs thereof.

For example, “pharmaceutically acceptable salts” refer to derivatives of the disclosed compounds wherein the specified compound is converted to an acid or base salt thereof. Such pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethane dislfonic, oxalic, isethionic, and the like.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The term “analogue” means a compound that comprises a chemically modified form of a specific compound or class thereof, and that maintains the pharmaceutical and/or pharmacological activities characteristic of said compound or class.

The term “prodrug”, as the term is used herein, is intended to include any covalently bonded carrier that releases an active drug agent of the present invention in vivo when such prodrug is administered to a mammalian subject. Because prodrugs are known to enhance numerous desirable qualities of pharmaceuticals (e.g., solubility, bioavailability, manufacturing, etc.) the pharmaceutical dosage forms of the present invention may contain compounds in prodrug form. Thus, the present invention is intended to cover prodrugs of the presently claimed compounds, methods of delivering the same, and compositions containing the same.

Examples of prodrugs for levodopa include 3-hydroxy-L-tyrosine ethyl ester and phenylglycine. In the dosage forms of the present invention, 3-hydroxy-L-tyrosine ethyl ester and/or phenylglycine can be used in combination with levodopa or as a replacement for levodopa in any of the formulations. Similarly, an appropriate prodrug for carbidopa can be used in combination with levodopa or as a replacement for carbidopa in any of the levodopa/carbidopa formulations of the present invention.

Prodrugs of the present invention may be prepared by modifying functional groups present in the compound in such a way that the modifications are cleaved, either in routine manipulation or in vivo, to the parent compound. The transformation in vivo may be, for example, as the result of some metabolic process, such as chemical or enzymatic hydrolysis of a carboxylic, phosphoric or sulphate ester, or reduction or oxidation of a susceptible functionality. Prodrugs within the scope of the present include compounds wherein a hydroxy, amino, or sulfhydryl group is bonded to any group that, when the prodrug of the present invention is administered to a mammalian subject, it cleaves to form a free hydroxyl, free amino, or free sulfydryl group respectively. Functional groups that may be rapidly transformed, by metabolic cleavage, in vivo form a class of groups reactive with the carboxyl group of the compounds of this invention. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkysilyl (such as trimethyl- and triethysilyl), monoesters formed with dicarboxylic acids (such as succinyl), and the like. Because of the ease with which metabolically cleavable groups of the compounds useful according to this invention are cleaved in vivo, the compounds bearing such groups act as prodrugs. The compounds bearing the metabolically cleavable groups have the advantage that they may exhibit improved bioavailability as a result of enhanced solubility and/or rate of absorption conferred upon the parent compound by virtue of the presence of the metabolically cleavable group.

A discussion of prodrugs is provided in the following: Design of Prodrugs, H. Bundgaard, ed., Elsevier, 1985; Methods in Enzymology, K. Widder et al., ed., Academic Press, 42, p.309-396, 1985; A Textbook of Drug Design and Development, Krogsgaard-Larsen and H. Bundgaard, ed., Chapter 5; “Design and Applications of Prodrugs” p.113-191, 1991; Advanced Drug Delivery Reviews, H. Bundgard, 8, p.1-38, 1992; Journal of Pharmaceutical Sciences, 77, p. 285, 1988; Chem. Pharm. Bull., N. Nakeya et al., 32, p. 692, 1984; Prodrugs as Novel Delivery Systems, T. Higuchi and V. Stella, Vol. 14 of the A.C.S. Symposium Series, and Bioreversible Carriers in Drug Design, Edward B. Roche, ed., American Pharmaceutical Association and Pergamon Press, 1987, each of which is incorporated herein by reference.

The term “derivative” means a chemically modified compound wherein the modification is considered routine by the ordinary skilled chemist, such as an ester or an amide of an acid, protecting groups, such as a benzyl group for an alcohol or thiol, and tert-butoxycarbonyl group for an amine.

The term “effective amount” means an amount of a compound/composition according to the present invention effective in producing the desired therapeutic effect.

Total daily dosages of the compounds useful according to this invention administered to a host in single or divided doses are generally in amounts of from about 0.01 mg/kg to about 100 mg/kg body weight daily, and preferably from about 0.05 mg/kg to about 50 mg/kg body weight daily. Both the levodopa and carbidopa doses fall within this mg/kg/day dosage range. The relative amounts of carbidopa and levodopa can vary from about 1:1 to about 1:50 in dosage forms according to the present invention. Other dosage ratios useful according to the present invention include carbidopa to levodopa ratios of 1:10, 5:26, 1:5, 1:4, 5:16, 1:3, 5:14, 1:2, 2:3, 3:4, or 5:6.

The skilled artisan will appreciate that daily dosages having an amount of active agent sufficient to treat Parkinson's disease will generally contain from about 25 mg to about 4,000 mg levodopa in combination with from about 5 mg to about 600 mg carbidopa. Dosage forms according to the present invention may also contain from about 25 or preferably 100 mg to about preferably 300 or 600 mg levodopa in combination with from about 12.5 or preferably 50 mg to about preferably 75 or 200 mg carbidopa. Preferred dosage forms contain 25, 37.5, 50, 70, 75, 80, 100, 125, 130, 150, 200, 250, 300, 400, or 600 mg levodopa and 12.5, 25, 37.5, 50, 62.5, 75, 100, 112.5, 125 or 150 mg carbidopa. Preferred dosage forms include all possible combinations of these amounts of levodopa and carbidopa. Dosage unit compositions may also contain amounts of levodopa and carbidopa in percentages of these dosages as may be used to make up the daily dose. It should be understood, however, that the specific dose level for any particular patient will depend upon a variety of factors including body weight, general health, gender, diet, time and route of administration, rates of absorption and excretion, combination with other drugs, and the severity of the particular disease being treated. Actual dosage levels of active ingredient in the compositions of the present invention may be varied so as to obtain an amount of active ingredient that is effective to obtain a desired therapeutic response for a particular composition and method of administration. The selected dosage level, therefore, depends upon the desired therapeutic effect, on the route of administration, on the desired duration of treatment, and other factors.

The dosage forms of the present invention are designed to administer active agent according to the combination of two release profiles. The first profile is an immediate release burst of carbidopa, another AAAD inhibitor (such as benserazide), or a combination of active ingredients such as an AAAD inhibitor and levodopa to provide early relief from symptoms via quick onset of effective blood plasma levels of active agent. Such early release is such that the in vitro dissolution rate of the immediate release component, according to measurements under the USP paddle method of 50 rpm in 900 ml aqueous buffer at pH 4 at 37° C. are from about 10% to about 99% levodopa released after 15 minutes and from about 75% to about 99% levodopa released after 1 hour.

The second profile is a controlled release profile in which the combination of active ingredients is released slowly over time to provide a plasma level effective to alleviate the symptoms of Parkinson's disease over a prolonged period. This controlled release profile may be over a period of 3, 4, 6, 8, 12, or 24 hours. Furthermore, the controlled release profile of the present invention is such that the in vitro dissolution rate of the controlled release component, according to measurements under the USP paddle method of 50 rpm in 900 ml aqueous buffer at pH 4 at 37° C., are from about 10% to about 60% levodopa released after 1 hour; from about 25% to about 80% released after 2 hours; from about 30% to about 85% levodopa released after 4 hours and from about 40% to about 99% levodopa released after about 6 hours, and chosen such that the peak plasma level of levodopa obtained in vivo occurs between 0.1 and 6 hours after administration of the dosage form.

The active ingredients of the present invention may be mixed with pharmaceutically acceptable carriers, diluents, adjuvants, excipients, or vehicles, such as preserving agents, fillers, polymers, disintegrating agents, glidants, wetting agents, emulsifying agents, suspending agents, sweetening agents, flavoring agents, perfuming agents, lubricating agents, acidifying agents, and dispensing agents, depending on the nature of the mode of administration and dosage forms. Such ingredients, including pharmaceutically acceptable carriers and excipients that may be used to formulate oral dosage forms, are described in the Handbook of Pharmaceutical Excipients, American Pharmaceutical Association (1986), incorporated herein by reference in its entirety. Pharmaceutically acceptable carriers include water, ethanol, polyols, vegetable oils, fats, waxes polymers, including gel forming and non-gel forming polymers, and suitable mixtures thereof. Examples of excipients include starch, pregelatinized starch, Avicel, lactose, milk sugar, sodium citrate, calcium carbonate, dicalcium phosphate, and lake blend. Examples of disintegrating agents include starch, alginic acids, and certain complex silicates. Examples of lubricants include magnesium stearate, sodium lauryl sulphate, talc, as well as high molecular weight polyethylene glycols. The artisan of ordinary skill in the art will recognize that many different excipients can be used in formulations according to the present invention and the list provided herein is not exhaustive.

Dosage forms can be made according to known methods in the art. Some preferred methods are described below.

Matrix Dosage Forms. The term matrix, as used herein, refers to a solid material having an active agent incorporated therein. Upon exposure to a dissolution media, channels are formed in the solid material so that the active agent can escape. Dosage forms according to one embodiment of the present invention may be in the form of coated or uncoated matrices. A coating, for example may contain immediate release carbidopa alone, or in the alternative, a combination of levodopa and carbidopa, and the matrix itself can contain the controlled release combination of levodopa and carbidopa.

The skilled artisan should appreciate that the matrix material can be chosen from a wide variety of materials that can provide the desired dissolution profiles. Materials can include, for example, one or more gel forming polymers such as polyvinyl alcohol, cellulose ethers including, for example, hydroxyl propyl alkyl, celluloses such as hydroxypropyl methyl cellulose, hydroxy alkyl celluloses such as hydroxy propyl cellulose, natural or synthetic gums such as guar gum, xanthum gum, and alginates, as well as, ethyl cellulose, polyvinyl pyrrolidone, fats, waxes, polycarboxylic acids or esters such as the Carbopo® series of polymers, methacrylic acid copolymers, and methacrylate polymers.

Methods of making matrix dosages are known in the art and any such method that can yield the desired immediate release and controlled release dissolution profiles may be relied upon according to the present invention. One such method involves the mixture of the levodopa and carbidopa combination with a solid polymeric material and one or more pharmaceutically acceptable excipients that are then blended and compressed in controlled release tablet cores. Such tablet cores can be used for further processing as bilayer tablets, press coated tablets, or film coated tablets.

A coating containing the immediate release carbidopa or carbidopa and levodopa in combination can be added to the outside of the controlled release tablet cores to produce a final dosage form. Such a coating can be prepared by mixing carbidopa alone, or a combination of levodopa and carbidopa, with polyvinylpyrrolidone (PVP) 29/32 or hydroxypropyl methylcellulose (HPMC) and water/isopropyl alcohol and triethyl acetate. Such an immediate release coating can be spray coated onto the tablet cores. The immediate release coating may also be applied using a press-coating process with a blend consisting of 80% by weight levodopa and carbidopa and 20% by weight of lactose and hydroxypropyl methylcellulose type 2910. Press coating techniques are known in the art and are described in U.S. Pat. No. 6,372,254 (Ting et al.), incorporated herein by reference in its entirety.

In addition, the formulation of respective release components can occur by appropriate granulation methods as is well known in the art. In wet granulation, solutions of the binding agent (polymer) are added with stirring to the mixed powders. The powder mass is wetted with the binding solution until the mass has the consistency of damp snow or brown sugar. The wet granulated material is forced through a sieving device. Moist material from the milling step is dried by placing it in a temperature controlled container. After drying, the granulated material is reduced in particle size by passing it through a sieving device. Lubricant is added, and the final blend is then compressed into a matrix dosage form.

In fluid-bed granulation, particles of inert material and/or active agent are suspended in a vertical column with a rising air stream. While the particles are suspended, a common granulating material in solution is sprayed into the column. There is a gradual particle buildup under a controlled set of conditions resulting in tablet granulation. Following drying and the addition of lubricant, the granulated material is ready for compression.

In dry-granulation, the active agent, binder, diluent, and lubricant are blended and compressed into tablets. The compressed large tablets are comminuted through the desirable mesh screen by sieving equipment. Additional lubricant is added to the granulated material and blended gently. The material is then compressed into tablets.

Particle Based Dosage Forms, Immediate Release Particles. The immediate release/controlled release dosage forms of the present invention can also take the form of pharmaceutical particles. The dosage forms can include immediate release particles in combination with controlled release particles in a ratio sufficient to deliver the desired dosages of active agents. The controlled release particles can be produced by coating the immediate release particles.

The particles can be produced according to any of a number of known methods for making particles. The immediate release particles comprise the active agent combination and a disintegrant. Suitable disintegrants include, for example, starch, low-substitution hydroxypropyl cellulose, croscarmellose sodium, calcium carboxymethyl cellulose, hydroxypropyl starch, and microcrystalline cellulose.

In addition to the above-mentioned ingredients, a controlled release matrix may also contain suitable quantities of other materials, for example, diluents, lubricants, binders, granulating aids, colorants, flavorants, and glidants that are conventional in the pharmaceutical arts. The quantities of these additional materials are sufficient to provide the desired effect to the desired formulation. A controlled release matrix incorporating particles may also contain suitable quantities of these other materials such as diluents, lubricants, binders, granulating aids, colorants, flavorants, and glidants that are conventional in the pharmaceutical arts in amounts up to about 75% by weight of the particulate, if desired.

Particles can assume any standard structure known in the pharmaceutical arts. Such structures include, for example, matrix particles, non-pareil cores having a drug layer and active or inactive cores having multiple layers thereon. A controlled release coating can be added to any of these structures to create a controlled release particle.

The term particle as used herein means a granule having a diameter of between about 0.01 mm and about 5.0 mm, preferably between about 0.1 mm and about 2.5 mm, and more preferably between about 0.5 mm and about 2 mm. The skilled artisan should appreciate that particles according to the present invention can be any geometrical shape within this size range and so long as the mean for a statistical distribution of particles falls within the particle sizes enumerated above, they will be considered to fall within the contemplated scope of the present invention.

The release of the therapeutically active agent from the controlled release formulation of the present invention can be further influenced, i.e., adjusted to a desired rate, by the addition of one or more release-modifying agents. The release-modifying agent may be organic or inorganic and include materials that can be dissolved, extracted, or leached from the coating in the environment of use. The pore-formers may comprise one or more hydrophilic materials such as hydroxypropyl methylcellulose. The release-modifying agent may also comprise a semi-permeable polymer. In certain preferred embodiments, the release-modifying agent is selected from hydroxypropyl methyclcellulose, lactose, metal stearates, and mixtures thereof.

In one preferred embodiment, oral dosage forms are prepared to include an effective amount of particles as described above within a capsule. For example, melt-extruded particles may be placed in a gelatin capsule in an amount sufficient to provide an effective controlled release dose when ingested and contacted by gastric fluid. In another preferred embodiment, a suitable amount of the particles are compressed into an oral tablet using conventional tableting equipment using standard techniques. Techniques and compositions for making tablets (compressed and molded), capsules (hard and soft gelatin), and pills are also described in Remington's Pharmaceutical Sciences, Arthur Osol, editor, pp. 1553-1593 (1980), incorporated herein by reference. The particles can be made by mixing the relevant ingredients and granulating the mixture. The resulting particles are dried and screened, and the particles having the desired size are used for drug formulation.

Controlled Release Particles. The controlled release particles of the present invention slowly release the combination of levodopa and carbidopa when ingested and exposed to gastric fluids, and then to intestinal fluids. The controlled release profile of the formulations of the invention can be altered, for example, by increasing or decreasing the thickness of the retardant coating, i.e., by varying the amount of overcoating. The resultant solid controlled release particles may thereafter be placed in a gelatin capsule in an amount sufficient to provide an effective controlled release dose when ingested and contacted by an environmental fluid, e.g., gastric fluid, intestinal fluid or dissolution media. The particles may be overcoated with an aqueous dispersion of a hydrophobic or hydrophilic material to modify the release profile. The aqueous dispersion of hydrophobic material preferably further includes an effective amount of plasticizer, e.g. triethyl citrate. Preformulated aqueous dispersions of ethylcellulose, such as Aquacoat® or Surelease®, may be used. If Surelease® is used, it is not necessary to separately add a plasticizer.

The hydrophobic material may be selected from the group consisting of alkylcellulose, acrylic and methacrylic acid polymers and copolymers, shellac, zein, hydrogenated castor oil, hydrogenated vegetable oil, or mixtures thereof. In certain preferred embodiments, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylicacid alkylamine copolymer, poly(methyl methacrylate), poly(methacrylic acid anhydride), polymethacrylate, polyacrylamide, poly(methacrylic acid anhydride), and glycidyl methacrylate copolymers. In alternate embodiments, the hydrophobic material is selected from materials such as one or more hydroxyalkyl celluloses such as hydroxypropyl methycellulose. The hydroxyalkyl cellulose is preferably a hydroxy (C₁ to C₆) alkyl cellulose, such as hydroxypropylcellulose, hydroxypropylmethylcellulose, or preferably hydroxyethylcellulose. The amount of the hydroxyalkyl cellulose in the present oral dosage form is determined, inter alia, by the precise rate of active agents desired and may vary from about 1% to about 80%.

In embodiments of the present invention where the coating comprises an aqueous dispersion of a hydrophobic polymer, the inclusion of an effective amount of a plasticizer in the aqueous dispersion of hydrophobic polymer can further improve the physical properties of the film. For example, because ethylcellulose has a relatively high glass transition temperature and does not form flexible films under normal coating conditions, it is necessary to plasticize the ethylcellulose before using it as a coating material. Generally, the amount of plasticizer included in a coating solution is based on the concentration of the film-former, e.g., most often from about 1 percent to about 50 percent by weight of the film-former. Concentration of the plasticizer, however, is preferably determined after careful experimentation with the particular coating solution and method of application.

Examples of suitable plasticizers for ethylcellulose include water-insoluble plasticizers such as dibutyl sebacate, diethyl phthalate, triethyl citrate, tributyl citrate, and triacetin, although other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to, citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for aqueous dispersions of ethyl cellulose. It has further been found that addition of a small amount of talc reduces the tendency of the aqueous dispersion to stick during processing and acts a polishing agent.

One commercially available aqueous dispersion of ethylcellulose is Aquacoat® which is prepared by dissolving the ethylcellulose in a water-immiscible organic solvent and then emulsifying the ethylcellulose in water in the presence of a surfactant and a stabilizer. After homogenization to generate submicron droplets, the organic solvent is evaporated under vacuum to form a pseudolatex. The plasticizer is not incorporated into the pseudolatex during the manufacturing phase. Thus, prior to using the pseudolatex as a coating, the Aquacoat® is mixed with a suitable plasticizer.

Another aqueous dispersion of ethylcellulose is commercially available as Surelease® (Colorcon, Inc., West Point, Pa., USA). This product is prepared by incorporating plasticizer into the dispersion during the manufacturing process. A hot melt of a polymer, plasticizer (dibutyl sebacate), and stabilizer (oleic acid) is prepared as a homogeneous mixture which is then diluted with an alkaline solution to obtain an aqueous dispersion which can be applied directly onto substrates.

In one preferred embodiment, the acrylic coating is an acrylic resin lacquer used in the form of an aqueous dispersion, such as that which is commercially available from Rohm Pharma under the trade name Eudragit®. In additional preferred embodiments, the acrylic coating comprises a mixture of two acrylic resin lacquers commercially available from Rohm Pharma under the trade names Eudragit® RL 30 D and Eudragit® RS 30 D. Eudragit® RL 30 D and Eudragit® RS 30 are copolymers of acrylic and methacrylic esters with a low content of quaternary ammonium groups, the molar ratio of ammonium groups to the remaining neutral (meth)acrylic esters being 1:20 in Eudragit® RL 30 and 1:40 in Eudragit® RS 30 D. The mean molecular weight is about 150,000 Daltons. The code designations RL (high permeability) and RS (low permeability) refer to the permeability properties of these agents. Eudragit® RL/RS mixtures are insoluble in water and in digestive fluids, however, coatings formed from them are swellable and permeable in aqueous solutions and digestive fluids.

The Eudragit® RL/RS dispersions may be mixed together in any desired ratio in order to ultimately obtain a controlled release formulation having a desirable dissolution profile. Desirable controlled release formulations may be obtained, for instance, from a retardant coating derived from one of a variety of coating combinations, such as 100% Eudragit® RL; 50% Eudragit® RL and 50% Eudragit® RS; or 10% Eudragit® RL and Eudragit® 90% RS. One skilled in the art should recognize that other acrylic polymers may also be used, for example, Eudragit®L. In addition to modifying the dissolution profile by altering the relative amounts of different acrylic resin lacquers, the dissolution profile of the ultimate product may also be modified, for example, by increasing or decreasing the thickness of the retardant coating.

In preferred embodiments of the present invention, the stabilized product is obtained by subjecting the coated substrate to oven curing at a temperature above the Tg of the plasticized acrylic polymer for the required time period, the optimum values for temperature and time for the particular formulation being determined experimentally. In certain embodiments of the present invention, the stabilized product is obtained via an oven curing conducted at a temperature of about 45° C. for a time period from about 1 to about 48 hours. It is also contemplated that certain products coated with the controlled release coating of the present invention may require a curing time longer than 24 to 48 hours, e.g., from about 48 to about 60 hours or more.

The coating solutions preferably contain, in addition to the film-former, plasticizer, and solvent system (i.e., water), a colorant to provide elegance and product distinction. Color may be added to the solution of the therapeutically active agent instead of, or in addition to the aqueous dispersion of hydrophobic material. For example, color may be added to Aquacoat® via the use of alcohol or propylene glycol based color dispersions, milled aluminum lakes and opacifiers such as titanium dioxide by adding color with shear to the water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retardant effect of the coating.

Spheroids or beads coated with the therapeutically active agents can be prepared, for example, by dissolving the therapeutically active agents in water and then spraying the solution onto a substrate, for example, non pareil 18/20 beads, using a Wuster insert. Optionally, additional ingredients are also added prior to coating the beads in order to assist the binding of the active agents to the beads, and/or to color the solution, etc. For example, a product that includes hydroxypropyl methycellulose with or without colorant (e.g., Opadry®, commercially available from Colorcon, Inc.) may be added to the solution and the solution mixed (e.g., for about 1 hour) prior to application onto the beads. The resultant coated substrate, beads in this example, may then be optionally overcoated with a barrier agent to separate the therapeutically active agent from the hydrophobic controlled release coating. An example of a suitable barrier agent is one that comprises hydroxypropylmethylcellulose. However, any film-former known in the art may be used. It is preferred that the barrier agent does not affect the dissolution rate of the final product.

Immediate release particles according to the present invention may be coated with a controlled release coating in order to change the release rate to obtain the dissolution rates according to the present invention.

Press Coated, Pulsatile Dosage Form. In another embodiment of the present invention, the carbidopa and levodopa combination is administered via a press coated pulsatile drug delivery system suitable for oral administration with a controlled release component, which contains a compressed blend of an active agent and one or more polymers, substantially enveloped by an immediate release component, which contains a compressed blend of the active agent and hydrophilic and hydrophobic polymers. The immediate release component preferably comprises a compressed blend of active agent and one or more polymers with disintegration characteristics such that the polymers disintegrate rapidly upon exposure to the aqueous medium.

The controlled release component preferably comprises a combination of hydrophilic and hydrophobic polymers. In this embodiment, once administered, the hydrophilic polymer dissolves away to weaken the structure of the controlled release component, and the hydrophobic polymer retards the water penetration and helps to maintain the shape of the drug delivery system.

In accordance with the present invention, the term “polymer” includes single or multiple polymeric substances, which can swell, gel, degrade or erode on contact with an aqueous environment (e.g., water). Examples include alginic acid, carboxymethylcellulose calcium, carboxymethylcellulose sodium, colloidal silicon dioxide, croscarmellose sodium, crospovidone, guar gum, magnesium aluminum silicate, methylcellulose, microcrystalline cellulose, polacrilin potassium, powdered cellulose, pregelatinized starch, sodium alginate, sodium starch glycolate, starch, ethylcellulose, gelatin, hydroxyethyl cellulose, hydroxypropyl cellulose, hydroxypropyl methylcellulose, polymethacrylates, povidone, pregelatinized starch, shellac, zein, and combinations thereof.

The term “hydrophilic polymers” as used herein includes one or more of carboxymethylcellulose, natural gums such as guar gum or gum acacia, gum tragacanth, or gum xanthan, hydroxyethyl cellulose, hydroxypropyl cellulose, hydroxypropyl methylcellulose, methylcellulose, and povidone, of which hydroxypropyl methylcellulose is further preferred. The term “hydrophilic polymers” can also include sodium carboxymethycellulose, hydroxymethyl cellulose, polyethelene oxide, hydroxyethyl methyl cellulose, carboxypolymethylene, polyethelene glycol, alginic acid, gelatin, polyvinyl alcohol, polyvinylpyrrolidones, polyacrylamides, polymethacrylamides, polyphosphazines, polyoxazolidines, poly(hydroxyalkylcarboxylic acids), an alkali metal or alkaline earth metal, carageenate alginates, ammonium alginate, sodium alganate, or mixtures thereof.

The hydrophobic polymer of the drug delivery system can be any hydrophobic polymer which will achieve the goals of the present invention including, but not limited to, one or more polymers selected from carbomer, carnauba wax, ethylcellulose, glyceryl palmitostearate, hydrogenated castor oil, hydrogenated vegetable oil type 1, microcrystalline wax, polacrilin potassium, polymethacrylates, or stearic acid, of which hydrogenated vegetable oil type 1 is preferred. Hydrophobic polymers can include, for example, a pharmaceutically acceptable acrylic polymer, including, but not limited to, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methyl methacrylate) copolymer, polyacrylamide, aminoalkyl methacrylate copolymer, poly(methacrylic acid anhydride), and glycidyl methacrylate copolymers. Additionally, the acrylic polymers may be cationic, anionic, or non-ionic polymers and may be acrylates, methacrylates, formed of methacrylic acid or methacrylic acid esters. The polymers may also be pH dependent.

The present invention also contemplates a method for preparing a press coated, pulsatile drug delivery system suitable for oral administration. This method includes the steps of combining an effective amount of an active agent, or a pharmaceutically acceptable salt thereof, and a polymer to form an immediate release component; combining an effective amount of an active agent, or a pharmaceutically acceptable salt thereof, and a combination of hydrophilic and hydrophobic polymers to form an controlled release component; and press coating the controlled release component to substantially envelop the immediate release component.

A preferred embodiment further includes the steps of combining an effective amount of an active agent, or a pharmaceutically acceptable salt thereof, and a polymer to form an immediate release component, and press coating the immediate release component to substantially envelop the controlled release component. In another preferred embodiment, the combining steps can be done by blending, wet granulation, fluid-bed granulation, or dry granulation according to methods recognized in the art.

The term “substantially envelop” is intended to define the total or near-total enclosure of a component. Such an enclosure includes, preferably, at least 80% enclosure, more preferably at least 90% enclosure, and most preferably at least 99% enclosure.

Without further elaboration, one skilled in the art having the benefit of the preceding description can utilize the present invention to the fullest extent. The following examples are illustrative only and do not limit the remainder of the disclosure in any way.

Example 1. The method described below was employed to obtain a press coated, pulsatile drug delivery system, the composition of which is set forth in Tables 1 and 2.

Appropriate weights of levodopa and carbidopa (weights shown in Tables 1 and 2) are intimately mixed for use in preparing immediate release and controlled release components of the formulations of the present invention.

Immediate release Component. The active agents are first mixed with silicon dioxide in a Patterson-Kelley V-blender for 10 minutes. Then microcrystalline cellulose and crosscarmellose sodium are added and blended for 10 more minutes. Finally, magnesium stearate is added to the blender and mixed for another 10 minutes. The powder blend is then compressed using a Manesty Dry Cota with a 0.2031 inch diameter, round, flat-face punch and die set. The hardness of the tablets is maintained at 4±2 kp.

Immediate release Component Plus Controlled release Component. The active agents are first mixed with silicon dioxide in a Patterson-Kelley V-blender for 10 minutes. Then hydroxypropyl methylcellulose 2208 and microcrystalline cellulose are added and blended for 10 more minutes. Finally, hydrogenated vegetable oil and magnesium stearate are added to the blender and mixed for another 10 minutes. The core tablets are press-coated using the Manesty Dry Cota with 0.3600″ in diameter, round, shallow concave punch and die set. The hardness of the tablets is maintained at 12±4 kp.

Example 2. Immediate release Component Plus Controlled release Component Plus Immediate release Component. The method of manufacture for the controlled release tablets is the same as described in Example 1. The application of the immediate release component was done by charging the controlled release tablets into a perforated pan coater or a fluidized particle coater and coating the tablet cores with a solution consisting of levodopa and carbidopa 80% w/lactose and hydroxypropyl methylcellulose type 2910.

For each IR or CR layer: All ingredients, except magnesium stearate are weighed and mixed thoroughly. The mixed ingredients are added to a high shear granulator and mixed for 5 minutes, with an impeller speed of 5 and a chopper speed of 4. Deionized water is employed as the granulating agent. Granules so made are dried in an oven overnight and then screened through a # 20 mesh (US standard). Oversize granules are milled, screened with the process repeated until all particles can be screened through a #20 mesh. The magnesium stearate is added to the screened particles and mixed thoroughly. Then the granules of each layer are compressed into bilayer tablets by a rotary tablet press.

TABLE 1

| | Quantity/Tablet | |
|---|---|---|
| | Example #1 | Example #2 |
| | RT-010 (press- | RT-011 (press- |
| | coated | coated |
| | w/o immediate | w/ immediate |
| | release coating) | release coating) |
| Immediate release (IR) Component | | |
| Levodopa/carbidopa 4:1 ratio 80% w/ | 50.0 mg | 50.0 mg |
| lactose | | |
| Croscarmellose sodium | 1.6 mg | 1.6 mg |
| Microcrystalline cellulose | 26.8 mg | 26.8 mg |
| Colloidal silicon dioxide | 0.8 mg | 0.8 mg |
| Magnesium stearate | 0.8 mg | 0.8 mg |
| Total: | 80.0 mg | 80.0 mg |
| IR Component Plus Controlled | | |
| Release (CR) Component | | |
| IR Component | 80.0 mg | 80.0 mg |
| Levodopa/carbidopa 4:1 ratio 80% w/ | 37.5 mg | 18.8 mg |
| lactose | | |
| Hydroxypropyl methylcellulose | 61.6 mg | 61.6 mg |
| type 2208 | | |
| Microcrystalline cellulose | 70.3 mg | 89.0 mg |
| Hydrogenated vegetable oil type 1 | 46.2 mg | 46.2 mg |
| Colloidal silicon dioxide | 2.2 mg | 2.2 mg |
| Magnesium stearate | 2.2 mg | 2.2 mg |
| Total: | 300.0 mg | 300.00 mg |
| IR Component Plus CR Component | | |
| Plus Immediate release Component | | |
| IR Component Plus ER Component | | 300.0 mg |
| Levodopa/carbidopa 4:1 ratio 80% w/ | | 18.7 mg |
| lactose | | |
| Hydroxypropyl methylcellulose | | 1.9 mg |
| type 2910 | | |
| Total: | | 320.6 mg |

TABLE 2
EXCIPIENT RANGE

| | Quantity/ | | |
|---|---|---|---|
| | tablet | | |
| | Example | | |
| | #1 RT-010 | | |
| | (press coated | | |
| | w/o IR coating) | Percent | Range |
| Immediate release | | | |
| Component | | | |
| Levodopa/carbidopa 4:1 ratio | 50.0 mg | 62.5% | |
| 80% w/lactose | | | |
| Croscarmellose sodium | 1.6 mg | 2.0% | 0.5-10.0% |
| Microcrystalline cellulose | 26.8 mg | 33.5% | 18.0-36.0% |
| Colloidal silicon dioxide | 0.8 mg | 1.0% | 0.5-2.0% |
| Magnesium stearate | 0.8 mg | 1.0% | 0.5-2.0% |
| Total: | 80.0 mg | | |
| Controlled release | | | |
| Component | | | |
| Levodopa/carbidopa 4:1 ratio | 37.5 mg | 17.0% | |
| 80% w/lactose | | | |
| Hydroxypropyl methylcellulose | 61.6 mg | 28.0% | 15.0-40.0% |
| type 2208 | | | |
| Microcrystalline cellulose | 70.3 mg | 32.0% | 8.0-57.0% |
| Hydrogenated vegetable oil | 46.2 mg | 21.0% | 10.0-30.0% |
| type 1 | | | |
| Colloidal silicon dioxide | 2.2 mg | 1.0% | 0.5-2.0% |
| Magnesium stearate | 2.2 mg | 1.0% | 0.5-2.0% |
| Total: | 220.0 mg | | |

Example 3. Example 3 employs the ingredients and amounts listed in Tables 3A, 3B, and 3C below for the formulations PX00502, PX03002, and PX03102, respectively. For each batch, whether 502, 3002 or 3102, the follows procedure is used: All ingredients, except magnesium stearate are weighed and mixed thoroughly. The mixed ingredients are added to a high shear granulator and mixed for 5 minutes, with an impeller speed of 5 and a chopper speed of 4. Deionized water is employed as the granulating agent. Granules so made are dried in an oven overnight and then screened through a # 20 mesh (US standard). Oversize granules are milled, screened with the process repeated until all particles can be screened through a #20 mesh. The magnesium stearate is added to the screened particles and mixed thoroughly. The resulting mixture can then be used for different types of dosage forms as set out in examples 4 and 5.

TABLE 3A

per tablet

| | | amount in |
|---|---|---|
| PX00502 | (w/w) % | mg |
| Carbidopa | 18 | 53.8 |
| Levodopa | 67 | 200.1 |
| Klucel | 12.9 | 38.5 |
| Lake blend | 0.3 | 0.9 |
| Mg stearate | 1.8 | 5.4 |
| Total | 100 | 298.7 |

TABLE 3B

per tablet

| | | amount in |
|---|---|---|
| PX03002 | (w/w) % | mg |
| Carbidopa | 11.3 | 27 |
| Levodopa | 41.9 | 100 |
| Avicel | 33.2 | 79.2 |
| Starch | 11.1 | 26.5 |
| Acdisol | 0.8 | 1.9 |
| Mg stearate | 1.7 | 3.8 |
| Total | 100 | 238.4 |

TABLE 3C

per tablet

| | | amount in |
|---|---|---|
| PX03102 | (w/w) % | mg |
| Carbidopa | 9.3 | 26.9 |
| Levodopa | 34.6 | 100.1 |
| Avicel | 27.4 | 79.3 |
| Starch | 27.4 | 79.3 |
| Mg Stearate | 1.3 | 3.8 |
| Total | 100 | 289.4 |

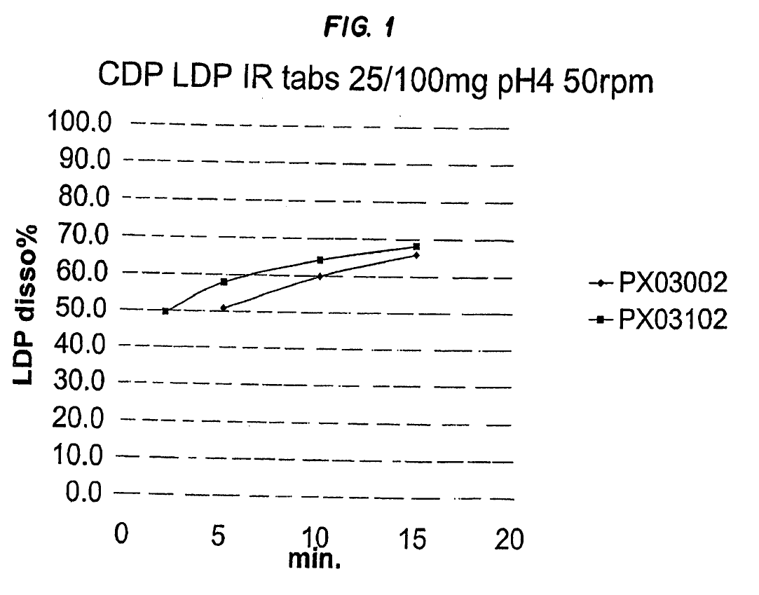

FIG. 1 shows the dissolution profiles of profiles of carbidopa/levodopa immediate release (IR) 25/100 mg formulations PX03002 and PX03102. As discussed above, all dissolution profiles were carried out by the standard USP paddle method of 50 rpm in 900 ml aqueous buffer at pH 4 at 37° C.

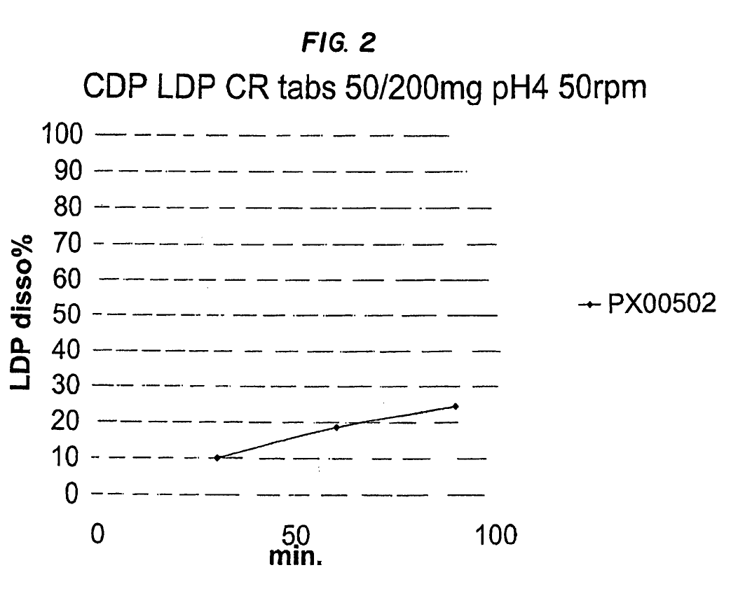

FIG. 2 shows the dissolution profile of a carbidopa/levodopa controlled release (CR) 50/200 mg formulation PX00502.

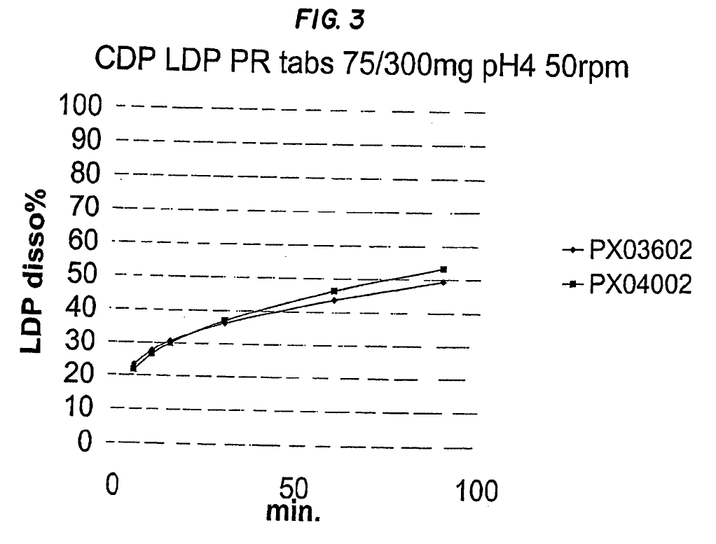

FIG. 3 shows the dissolution profiles of carbidopa/levodopa 75/300 mg formulations PX03602 and PX04002. Note that controlled release (or prolonged release (PR)) tablets PX03602 comprise the combination of PX0502(CR) and PX03102, and PR tablets PX04002 comprise the combination of PX0502(CR) and PX03002.

Example 4. The lot 3102 particles produced in Example 3 are segregated into two equal portions of 125 grams each. One portion is coated in a fluidized pan with a mixture of 24.25 g of PVP 29/32, 1000 g of deionized water and isopropyl alcohol (15%), and 0.75 g of triethyl acetate. The particles are dried and thoroughly mixed with the uncoated particles. The particle mixture is then loaded into immediate release gelatin capsules.

Example 5. Particles produced according to lots 3002 and 502 of Example 3 are loaded into the two separate hoppers of a dual layer tablet punch. The punch is actuated and two-layer tablets are produced.

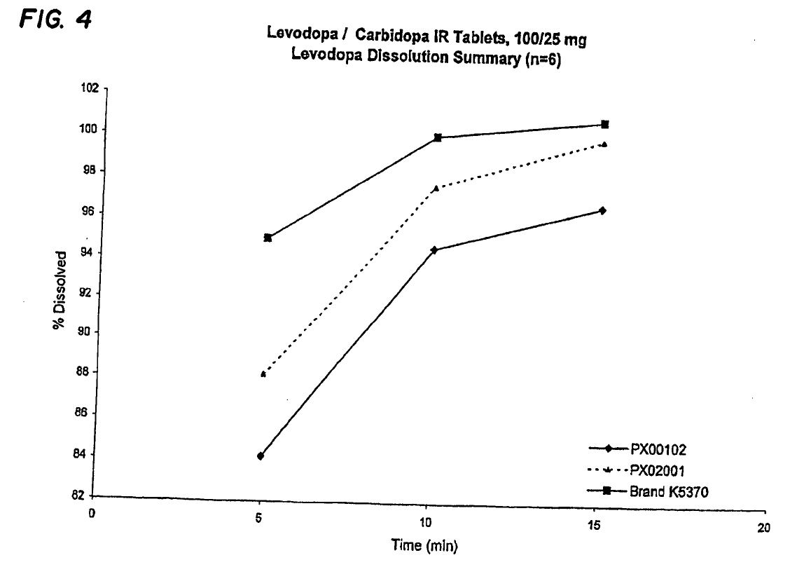

Example 6. The dissolution summaries for carbidopa/levodopa immediate release (IR) 25/100 mg formulations PX00102, PX02001, and Brand K5370 are shown in Tables 4, 5, and 6, respectively. All data was obtained according to measurements under the USP paddle method of 50 rpm in 900 ml at pH 1.2 (0.1 N HCl) at 37° C. FIG. 4 is a graph of the dissolution profiles of carbidopa/levodopa immediate release (IR) 25/100 mg formulations PX00102, PX02001, and Brand K5370.

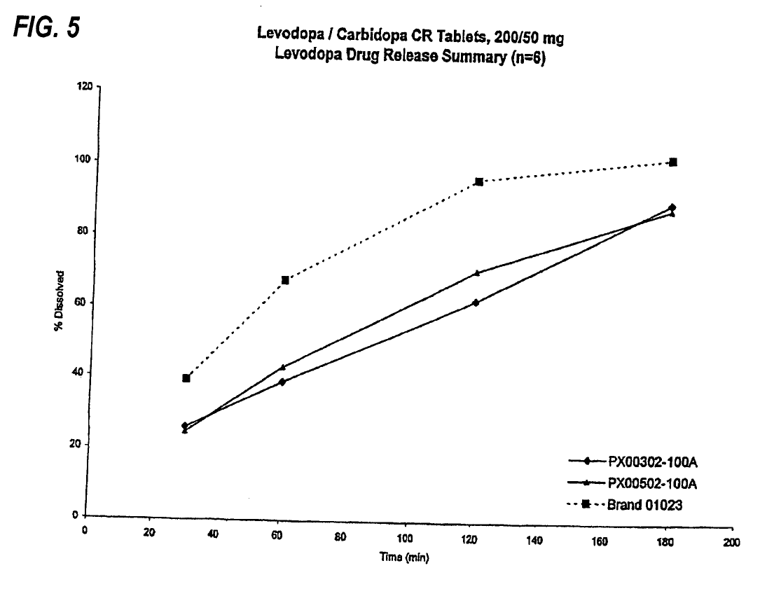

Example 7. The dissolution summaries for carbidopa/levodopa controlled release (CR) 50/200 mg formulations PX00302, PX00502, and Brand 01023 are shown in Tables 7, 8, and 9, respectively. All data was obtained according to measurements under the USP paddle method of 50 rpm in 900 ml at pH 1.2 (0.1 N HCl) at 37° C. FIG. 5 is a graph of the dissolution of carbidopa/levodopa controlled release (CR) 50/200 mg formulations PX00302, PX00502, and Brand 01023.

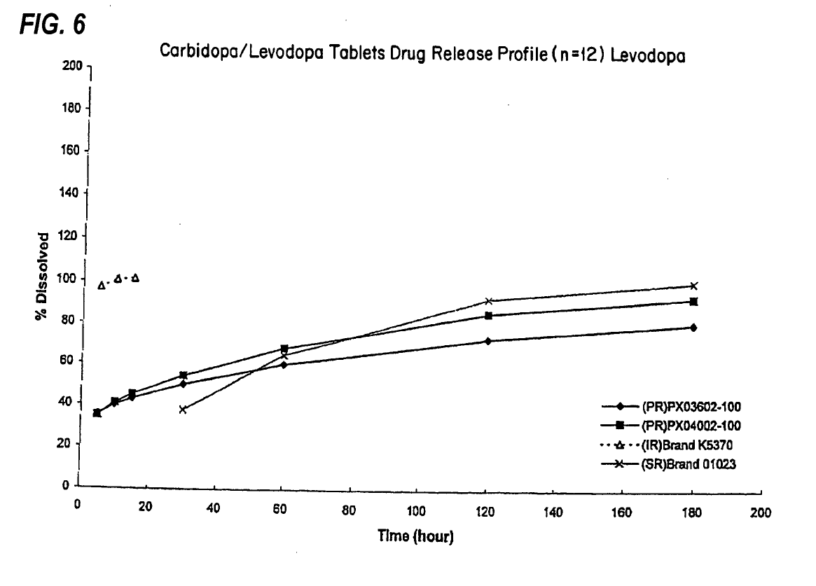

Example 8. The dissolution summaries for carbidopa/levodopa formulations PX03602 (controlled release, 75/300 mg), PX04002 (controlled release, 75/300 mg), Brand K5370 (immediate release, 25/100 mg), and Brand 01023 (controlled release, 50/200 mg) are shown in Tables 10, 11, 12, and 13, respectively. All data was obtained according to measurements under the USP paddle method of 50 rpm in 900 ml at pH 1.2 (0.1 N HCl) at 37° C. FIG. 6 is a graph of the dissolution profiles of carbidopa/levodopa formulations PX03602 (controlled release, 75/300 mg), PX04002 (controlled release, 75/300 mg), Brand K5370 (immediate release, 25/100 mg), and Brand 01023 (controlled release, 50/200 mg). As note in Example 3, controlled release (or prolonged release (PR)) tablets PX03602 comprise the combination of PX0502(CR) and PX03102, and PR tablets PX04002 comprise the combination of PX0502(CR) and PX03002.

Example 9. Pharmaceutical dosage forms containing an AAAD inhibitor, levodopa, and a COMT inhibitor, and employing the ingredients and amounts listed in Table 3D below may be produced. All ingredients, except magnesium stearate are weighed and mixed thoroughly. The mixed ingredients are added to a high shear granulator and mixed for 5 minutes, with an impeller speed of 5 and a chopper speed of 4. Deionized water is employed as the granulating agent. Granules so made are dried in an oven overnight and then screened through a # 20 mesh (US standard). Oversize granules are milled, screened with the process repeated until all particles can be screened through a #20 mesh. The magnesium stearate is added to the screened particles and mixed thoroughly. Then the granules of each layer are compressed into bilayer tablets by a rotary tablet press.

TABLE 3D

| Ingredients | weight (mg) |
|---|---|
| Immediate release component | |
| Carbidopa, USP | 27.04 |
| Levodopa, USP | 100.00 |
| Entacapone | 200.00 |
| Microcrystalline Cellulose, NF | 22.22 |
| Starch, NF | 4.49 |
| Croscarmellose Sodium, NF | 4.49 |
| Crospovidone, NF | 2.94 |
| Magnesium Stearate, NF | 2.21 |
| Controlled release component | |
| Carbidopa, USP | 27.0 |
| Levodopa, USP | 100.02 |
| Hydroxypropyl Cellulose, NF | 19.25 |
| Lake Blend Purple | 0.38 |
| Magnesium Stearate, NF | 2.70 |
| Total tablet weight | 512.74 |

### Data For FIG. 4

### Levodopa/Carbidopa IR Tablets, 100/25 mg

### Levodopa Dissolution Summary (n=6)

SGF/37° C./50 rpm/Paddle

TABLE 4
Lot PX00102-100 T = 0

| Time | % Dissolved | | | | | | | | | | | | Range | | | |
|---|---|---|---|---|---|---|---|---|---|---|---|---|---|---|---|---|
| (min) | V1 | V2 | V3 | V4 | V5 | V6 | V7 | V8 | V9 | V10 | V11 | V12 | Min | Max | Mean | SD |
| 5 | 88 | 82 | 90 | 83 | 89 | 89 | 85 | 87 | 85 | 85 | 68 | 79 | 68 | 90 | 84 | 6.03 |
| 10 | 96 | 90 | 95 | 91 | 95 | 99 | 94 | 96 | 95 | 99 | 89 | 96 | 89 | 99 | 95 | 3.18 |
| 15 | 98 | 92 | 96 | 93 | 97 | 100 | 96 | 97 | 99 | 101 | 91 | 100 | 91 | 101 | 97 | 3.2 |

TABLE 5
Lot PX02001-100 T = 0

| Time | % Dissolved | | | | | | | | | | | | Range | | | |
|---|---|---|---|---|---|---|---|---|---|---|---|---|---|---|---|---|
| (min) | V1 | V2 | V3 | V4 | V5 | V6 | V7 | V8 | V9 | V10 | V11 | V12 | Min | Max | Mean | SD |
| 5 | 87 | 96 | 83 | 95 | 80 | 97 | 87 | 89 | 84 | 82 | 90 | 88 | 80 | 97 | 88 | 5.57 |
| 10 | 98 | 98 | 97 | 101 | 92 | 100 | 98 | 98 | 98 | 93 | 98 | 100 | 92 | 101 | 98 | 2.64 |
| 15 | 100 | 99 | 100 | 103 | 97 | 101 | 100 | 100 | 101 | 97 | 100 | 101 | 97 | 103 | 100 | 1.68 |

TABLE 6
Brand (Sinemet, exp. 02/05) Lot K5370 T = 0

| Time | % Dissolved | | | | | | | | | | | | Range | | | |
|---|---|---|---|---|---|---|---|---|---|---|---|---|---|---|---|---|
| (min) | V1 | V2 | V3 | V4 | V5 | V6 | V7 | V8 | V9 | V10 | V11 | V12 | Min | Max | Mean | SD |
| 5 | 92 | 89 | 97 | 97 | 93 | 97 | 94 | 95 | 92 | 99 | 93 | 102 | 89 | 102 | 95 | 3.62 |
| 10 | 99 | 95 | 100 | 100 | 99 | 102 | 101 | 99 | 99 | 101 | 101 | 104 | 95 | 104 | 100 | 2.05 |
| 15 | 100 | 97 | 101 | 101 | 100 | 103 | 102 | 100 | 101 | 101 | 101 | 104 | 97 | 104 | 101 | 1.69 |

Data For FIG. 5

### Levodopa/Carbidopa CR Tablets, 200/50 mg

### Levodopa Dissolution Summary (n=6)

### SGF/37° C./50 rpm/Paddle

TABLE 7
PX00302-100A T = 0

| Time | % Dissolved | | | | | | | | | | | | Range | | | |
|---|---|---|---|---|---|---|---|---|---|---|---|---|---|---|---|---|
| (min) | V1 | V2 | V3 | V4 | V5 | V6 | V7 | V8 | V9 | V10 | V11 | V12 | Min | Max | Mean | SD |
| 30 | 26 | 26 | 28 | 26 | 25 | 25 | 24 | 26 | 26 | 26 | 26 | 26 | 24 | 28 | 26 | 1.07 |
| 60 | 40 | 39 | 41 | 39 | 37 | 39 | 36 | 38 | 39 | 39 | 39 | 39 | 36 | 41 | 39 | 1.21 |
| 120 | 58 | 62 | 74 | 63 | 56 | 66 | 56 | 57 | 70 | 65 | 58 | 65 | 56 | 74 | 62 | 5.87 |
| 180 | 83 | 90 | 101 | 92 | 75 | 97 | 82 | 87 | 98 | 78 | 93 | 100 | 75 | 101 | 90 | 8.82 |

TABLE 8
PX00502-100A T = 0

| Time | % Dissolved | | | | | | | | | | | | Range | | | |
|---|---|---|---|---|---|---|---|---|---|---|---|---|---|---|---|---|
| (min) | V1 | V2 | V3 | V4 | V5 | V6 | V7 | V8 | V9 | V10 | V11 | V12 | Min | Max | Mean | SD |
| 30 | 23 | 24 | 24 | 26 | 25 | 24 | 24 | 25 | 25 | 25 | 24 | 24 | 23 | 26 | 24 | 0.82 |
| 60 | 40 | 43 | 43 | 44 | 45 | 42 | 43 | 44 | 43 | 44 | 42 | 42 | 40 | 45 | 43 | 1.40 |
| 120 | 67 | 71 | 70 | 72 | 75 | 68 | 70 | 73 | 71 | 72 | 69 | 69 | 67 | 75 | 71 | 2.17 |
| 180 | 84 | 88 | 88 | 88 | 91 | 84 | 90 | 93 | 89 | 88 | 86 | 88 | 84 | 93 | 88 | 2.46 |

TABLE 9
Brand 01023 T = 0

| Time | % Dissolved | | | | | | | | | | | | Range | | | |
|---|---|---|---|---|---|---|---|---|---|---|---|---|---|---|---|---|
| (min) | V1 | V2 | V3 | V4 | V5 | V6 | V7 | V8 | V9 | V10 | V11 | V12 | Min | Max | Mean | SD |
| 30 | 37 | 47 | 42 | 42 | 42 | 34 | 42 | 41 | 30 | 41 | 37 | 34 | 30 | 47 | 39 | 4.81 |
| 60 | 64 | 79 | 71 | 71 | 74 | 59 | 75 | 69 | 53 | 71 | 66 | 60 | 53 | 79 | 68 | 7.69 |
| 120 | 92 | 101 | 99 | 99 | 99 | 93 | 102 | 98 | 84 | 97 | 97 | 93 | 84 | 102 | 96 | 4.86 |
| 180 | 101 | 103 | 103 | 102 | 102 | 105 | 103 | 101 | 103 | 100 | 102 | 104 | 100 | 105 | 102 | 1.55 |

### Data For FIG. 6

### Levodopa/Carbidopa Compositions

### Drug Release Summary (n=12), SGF/37° C./50 rpm/Paddle

TABLE 10
(PR, 75/300 mg) PX03602-100

| Time | % Dissolved | | | | | | | | | | | | Range | | | |
|---|---|---|---|---|---|---|---|---|---|---|---|---|---|---|---|---|
| (min) | V1 | V2 | V3 | V4 | V5 | V6 | V7 | V8 | V9 | V10 | V11 | V12 | Min | Max | Mean | SD |
| 5 | 34.6 | 39.9 | 35.3 | 33.2 | 38.0 | 37.6 | 42.2 | 32.1 | 28.6 | 33.6 | 38.3 | 33.6 | 29 | 42 | 36 | 3.8 |
| 10 | 39.1 | 45.0 | 38.9 | 36.9 | 41.3 | 41.4 | 47.7 | 36.7 | 33.3 | 37.5 | 42.4 | 39.0 | 33 | 48 | 40 | 3.9 |
| 15 | 42.0 | 49.0 | 41.2 | 39.2 | 43.5 | 44.7 | 51.5 | 40.1 | 36.7 | 40.1 | 45.3 | 42.8 | 37 | 52 | 43 | 4.2 |
| 30 | 48.8 | 59.0 | 45.9 | 44.4 | 48.4 | 51.3 | 60.2 | 47.8 | 42.6 | 46.7 | 52.5 | 51.5 | 43 | 60 | 50 | 5.4 |
| 60 | 55.5 | 75.9 | 52.6 | 51.9 | 55.6 | 61.8 | 72.0 | 59.4 | 51.2 | 56.7 | 63.2 | 64.7 | 51 | 76 | 60 | 7.9 |
| 120 | 65.8 | 98.9 | 61.9 | 62.4 | 65.5 | 72.3 | 82.5 | 74.7 | 63.3 | 70.6 | 76.9 | 81.5 | 62 | 99 | 73 | 10.9 |
| 180 | 73.7 | 102.2 | 68.2 | 69.1 | 72.1 | 80.1 | 88.2 | 83.6 | 70.7 | 79.5 | 86.9 | 91.0 | 68 | 102 | 80 | 10.4 |

TABLE 11
(PR, 75/300 mg) PX04002-100

| Time | % Dissolved | | | | | | | | | | | | Range | | | |
|---|---|---|---|---|---|---|---|---|---|---|---|---|---|---|---|---|
| (min) | V1 | V2 | V3 | V4 | V5 | V6 | V7 | V8 | V9 | V10 | V11 | V12 | Min | Max | Mean | SD |
| 5 | 35.1 | 34.0 | 27.3 | 29.2 | 30.4 | 24.0 | 33.7 | 33.5 | 36.3 | 33.8 | 36.3 | 35.6 | 24 | 36 | 35 | 1.3 |
| 10 | 40.6 | 38.5 | 32.0 | 33.8 | 37.4 | 29.5 | 39.8 | 38.5 | 42.4 | 41.8 | 40.8 | 41.7 | 30 | 42 | 41 | 1.5 |
| 15 | 44.2 | 41.4 | 35.1 | 36.8 | 42.2 | 32.8 | 43.9 | 41.9 | 46.6 | 46.9 | 44.4 | 46.1 | 33 | 47 | 45 | 1.9 |
| 30 | 52.3 | 47.3 | 41.4 | 43.2 | 52.5 | 39.2 | 52.6 | 49.5 | 56.0 | 57.7 | 53.0 | 55.3 | 39 | 58 | 54 | 2.9 |
| 60 | 64.7 | 56.1 | 51.0 | 52.7 | 66.8 | 48.7 | 64.9 | 61.2 | 70.6 | 75.0 | 66.9 | 69.8 | 49 | 75 | 68 | 4.8 |
| 120 | 79.3 | 68.8 | 64.4 | 71.4 | 84.6 | 63.1 | 80.4 | 78.2 | 89.9 | 92.0 | 84.9 | 88.0 | 63 | 92 | 86 | 5.4 |
| 180 | 87.1 | 77.5 | 73.2 | 75.4 | 93.5 | 72.0 | 89.0 | 88.8 | 96.3 | 96.2 | 93.9 | 94.1 | 72 | 96 | 93 | 3.4 |

TABLE 12
(IR 25/100 mg) Brand Lot K5370

| | | % Dissolved | | | | | | | | | | | | Range | | | |
|---|---|---|---|---|---|---|---|---|---|---|---|---|---|---|---|---|---|
| Time | | V1 | V2 | V3 | V4 | V5 | V6 | V7 | V8 | V9 | V10 | V11 | V12 | Min | Max | Mean | SD |
| 5 | min | 100.8 | 95.9 | 94.6 | 99.1 | 96.5 | 97.1 | 97.6 | 93.0 | 99.2 | 100.8 | 93.7 | 98.8 | 93 | 101 | 97 | 2.6 |
| 10 | min | 101.7 | 99.3 | 100.3 | 102.6 | 100.9 | 100.9 | 99.7 | 98.3 | 101.9 | 103.4 | 98.3 | 100.5 | 98 | 103 | 101 | 1.6 |
| 15 | min | 101.6 | 100.7 | 100.7 | 103.1 | 101.8 | 101.3 | 100.7 | 100.7 | 102.0 | 103.6 | 99.6 | 100.7 | 100 | 104 | 101 | 1.1 |

TABLE 13
(SR 50/200 mg) Brand Lot 01023

| Time | % Dissolved | | | | | | | | | | | | Range | | | |
|---|---|---|---|---|---|---|---|---|---|---|---|---|---|---|---|---|
| (min) | V1 | V2 | V3 | V4 | V5 | V6 | V7 | V8 | V9 | V10 | V11 | V12 | Min | Max | Mean | SD |
| 30 | 45.9 | 41.2 | 36.5 | 39.0 | 36.3 | 35.7 | 40.5 | 36.7 | 36.5 | 42.5 | 30.3 | 30.5 | 30 | 46 | 38 | 4.6 |
| 60 | 77.2 | 72.0 | 62.3 | 65.9 | 61.5 | 60.6 | 69.7 | 61.9 | 62.0 | 74.7 | 53.4 | 54.3 | 53 | 77 | 65 | 7.5 |
| 120 | 98.9 | 98.9 | 91.7 | 94.1 | 89.1 | 88.8 | 95.7 | 90.2 | 89.8 | 103.9 | 85.7 | 85.4 | 85 | 104 | 93 | 5.7 |
| 180 | 101.3 | 103.1 | 101.4 | 100.8 | 99.2 | 98.1 | 99.2 | 99.7 | 99.4 | 104.6 | 101.3 | 99.1 | 98 | 105 | 101 | 1.9 |

What is claimed is:

1. A pharmaceutical dosage form comprising an immediate release and a controlled release component,
wherein said immediate release component and said controlled release component each comprises an AAAD inhibitor and levodopa in a ratio of from about 1:1 to about 1:50;
wherein said immediate release component exhibits an in vitro dissolution profile comprising at least about 10% levodopa release after 15 minutes and at least about 60% levodopa release after 1 hour;
and wherein said controlled release component exhibits an in vitro dissolution profile comprising from about 10% to about 60% levodopa release after 1 hour, from about 20% to about 80% levodopa release after 2 hours, and at least about 30% levodopa release after 6 hours.

2. A pharmaceutical dosage form according to claim 1 wherein said dosage form further comprises a COMT inhibitor.

3. A pharmaceutical dosage form according to claim 1 wherein said AAAD inhibitor is carbidopa.

4. A pharmaceutical dosage form according to claim 1 wherein said levodopa is in the form (−)-L-α-amino-β-(3,4-dihydroxybenzene) propanoic acid, 3-hydroxy-L-tyrosine ethyl ester, phenylglycine, or a mixture thereof.

5. A pharmaceutical dosage form according to claim 1 wherein said dosage form exhibits an in vivo plasma profile comprising a levodopa release peak from about 6 minutes to about 6 hours after administration to a fasting patient.

6. A pharmaceutical dosage form according to claim 1 wherein said dosage form exhibits an in vivo plasma profile comprising a levodopa release peak from about 6 minutes to about 5 hours after administration to a fasting patient.

7. A pharmaceutical dosage form according to claim 2 wherein said dosage form exhibits an in vivo plasma profile comprising a COMT inhibitor release peak from about 6 minutes to about 6 hours after administration to a fasting patient.

8. A pharmaceutical dosage form according to claim 2 wherein said dosage form exhibits an in vivo plasma profile comprising a COMT inhibitor release peak from about 6 minutes to about 5 hours after administration to a fasting patient.

9. A pharmaceutical dosage form according to claim 2 wherein said dosage form comprises up to about 1000 mg COMT inhibitor.

10. A pharmaceutical dosage form according to claim 2 wherein said dosage form comprises from about 20 mg to about 500 mg COMT inhibitor.

11. A pharmaceutical dosage form according to claim 2 wherein said dosage form comprises from about 50 mg to about 500 mg COMT inhibitor.

12. A pharmaceutical dosage form according to claim 2 wherein said dosage form comprises from about 100 mg to about 200 mg COMT inhibitor.

13. A pharmaceutical dosage form according to claim 2 wherein said COMT inhibitor is contained only within said immediate release component.

14. A pharmaceutical dosage form according to claim 2 wherein said COMT inhibitor is contained only within said controlled release component.

15. A pharmaceutical dosage form according to claim 2 wherein said COMT inhibitor is contained within both said immediate release and said controlled release components.

16. A pharmaceutical dosage form according to claim 3 wherein said immediate release component comprises carbidopa and levodopa in a ratio of from about 1:1 to about 1:10.

17. A pharmaceutical dosage form according to claim 3 wherein said immediate release component comprises carbidopa and levodopa in a ratio of from about 1:1 to about 1:5.

18. A pharmaceutical dosage form according to claim 3 wherein said immediate release component comprises carbidopa and levodopa in a ratio of from about 1:1 to about 1:4.

19. A pharmaceutical dosage form according to claim 3 wherein said controlled release component comprises carbidopa and levodopa in a ratio of from about 1:1 to about 1:10.

20. A pharmaceutical dosage form according to claim 3 wherein said controlled release component comprises carbidopa and levodopa in a ratio of from about 1:1 to about 1:5.

21. A pharmaceutical dosage form according to claim 3 wherein said controlled release component comprises carbidopa and levodopa in a ratio of from about 1:1 to about 1:4.

22. A pharmaceutical dosage form according to claim 1 wherein said immediate release component exhibits an in vitro dissolution profile comprising at least about 10% levodopa release after 15 minutes and at least about 95% levodopa release after 1 hour.

23. A pharmaceutical dosage form according to claim 2 wherein said COMT inhibitor is selected from the group consisting of CGP-28014, entacapone, and tolcapone.

24. A pharmaceutical dosage form according to claim 1 wherein said dosage form further comprises one or more drugs selected from the group consisting of anti-cholinergics, beta 2-agonists, cyclooxygenase-2 (COX-2) inhibitors, dopamine receptor agonists, monoamine oxidase (MAO) inhibitors, opiate delta receptor agonists, opiate delta receptor antagonists, and N-methyl-D-aspartate (NMDA) antagonists.

25. A pharmaceutical dosage form according to claim 1 wherein said dosage form further comprises one or more drugs selected from the group consisting of albuterol, alpha-lipoic acid, amantadine, andropinirole, apomorphine, baclofen, biperiden, benztropine, bromocriptine, budipine, cabergoline, clozapine, deprenyl, dextromethorphan, dihydroergokryptine, dihydrolipoic acid, eliprodil, eptastigmine, ergoline, formoterol, galanthamine, lazabemide, lysuride, mazindol, memantine, mofegiline, orphenadrine, pergolide, pirbuterol, pramipexole, propentofylline, procyclidine, rasagiline, remacemide, riluzole, rimantadine, ropinirole, salmeterol, selegiline, spheramine, terguride, and trihexyphenidyl.

26. A pharmaceutical dosage form according to claim 3 wherein said immediate release component comprises from about 2.5 mg to about 75 mg carbidopa and from about 25 mg to about 300 mg levodopa.

27. A pharmaceutical dosage form according to claim 3 wherein said controlled release component comprises from about 2.5 mg to about 200 mg carbidopa and from about 25 mg to about 600 mg levodopa.

28. A pharmaceutical dosage form according to claim 3 wherein the controlled release component comprises from about 5 mg to about 150 mg carbidopa and from about 50 mg to about 500 mg levodopa.

29. A pharmaceutical dosage form according to claim 3 wherein the controlled release component comprises from about 12.5 mg to about 50 mg carbidopa and from about 50 mg to about 200 mg levodopa.

30. A pharmaceutical dosage form according to claim 1 wherein said dosage form is a particle.

31. A pharmaceutical dosage form according to claim 1 wherein said dosage form is a tablet.

32. A pharmaceutical dosage form according to claim 31 wherein said tablet is a layered tablet.

33. A method of treating a patient suffering from a pathology or disease characterized by reduced dopamine levels in the brain comprising the step of administering a pharmaceutical dosage form according to claim 1.
